(19) United States
(12) Patent Application Publication
(10) Pub. No.: US 2010/0028425 A1
(43) Pub. Date: Feb. 4, 2010

(54) PHARMACEUTICAL COMPOSITION OF ATOVAQUONE

(75) Inventors: Kamal Mehta, Rajasthan (IN); Sanjay Pandurang Lade, Navi Mumbai Maharashtra (IN); Srinivas G. Arra, Navi Mumbai Maharashtra (IN)

Correspondence Address: GLENMARK PHARMACEUTICALS INC USA, 750 CORPORATE DRIVE, MAHWAH, NJ, 07430, US

(73) Assignee: GLENMARK GENERICS LTD., Mumbai (IN)

(21) Appl. No.: 12/507,185

(22) Filed: Jul. 22, 2009

Related U.S. Application Data
(60) Provisional application No. 61/179,032, filed on May 18, 2009.

(30) Foreign Application Priority Data
Jul. 31, 2008 (IN) 1637/MUM/2008

(51) Int. Cl. A61K 9/20; A61K 9/14; A61K 31/12; A61K 31/155; A61P 33/06

(52) U.S. Cl. 424/464; 424/489; 514/682; 514/636

(57) ABSTRACT

The present invention relates to immediate release pharmaceutical compositions for oral administration comprising micronized atovaquone particles and at least one drug, wherein about 90% of the atovaquone particles have a volume diameter between about 4 μm to about 8 μm; and having a uniform release profile after a storage for at least three months at 40° C. and 75% relative humidity.

### PRIORITY

This application claims the benefit to Indian Provisional Application 1637/MUM/2008, filed on Jul. 31, 2008, and under 35 U.S.C. §119 to U.S. Provisional Application 61/179032, filed May 18, 2009, the contents of each which are incorporated by reference herein.

### BACKGROUND OF THE INVENTION

1. Technical Field

The present invention relates to immediate release pharmaceutical compositions for oral administration comprising micronized atovaquone particles and at least one drug, wherein about 90% of the atovaquone particles have a volume diameter between about 4 μm to about 8 μm.

2. Description of the Related Art

Atovaquone is an anti-pneumocystic drug disclosed in European Patent No. 0123238 and U.S. Pat. No. 5,053,432.

The chemical name of atovaquone is trans 2-[4-(4-chlorophenyl)cyclohexyl]-3-hydroxy-1,4-naphthalenedione. Atovaquone is a yellow crystalline solid that is practically insoluble in water. It has a molecular weight of 366.84 and the molecular formula C₂₂H₁₉ClO₃. The compound has the following structural formula:

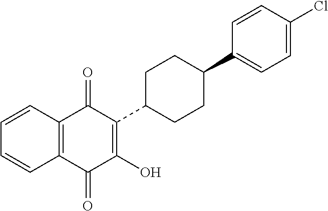

Atovaquone is available in the market under the trade names Mepron®, Malarone Pediatric®, Malarone®, is available in the form of oral suspension (750 mg/5 ml). Malarone Pediatric® and Malarone® are the combination products of atovaquone with proguanil hydrochloride which are available in tablet form of (25 mg proguanil HCl, 62.5 mg atovaquone and 100 mg proguanil HCl, 250 mg atovaquone, respectively). Malarone® is indicated for the prophylaxis of P. falciparum malaria.

European Patent No. 0362996 discloses the use of atovaquone in the treatment and/or prophylaxis of Pneumocystis carinii pneumoni, using formulations suitable for pulmonary administration containing naphthoquinone particles having a diameter in the range 0.5 to 7 μm.

European Patent Nos. 0445141 and 0496729 disclose the uses of atovaquone against toxoplasmosis and tryptosporidiosis, respectively.

U.S. Pat. No. 6,018,080 discloses microfluidized particles of atovaquone, a method for their preparation and a pharmaceutical composition containing the same and its use in therapy.

U.S. Pat. Nos. 6,018,080 and 6,649,659 disclose atovaquone particles having particle sizes in the range of 0.1-3 μm and the method of their preparation using a microfluidizer.

### SUMMARY OF THE INVENTION

The present invention provides immediate release pharmaceutical compositions for oral administration comprising micronized atovaquone particles and at least one drug, wherein about 90% of the atovaquone particles have a volume diameter between about 3 μm to about 10 μm.

The present invention provides immediate release pharmaceutical compositions for oral administration comprising micronized atovaquone particles, and at least one drug, wherein about 90% of the atovaquone particles have a volume diameter between about 3 μm to about 10 μm and wherein the drug is proguanil hydrochloride.

The present invention provides immediate release pharmaceutical compositions for oral administration comprising micronized atovaquone particles and at least one drug, wherein about 90% of the atovaquone particles have a volume diameter between about 3 μm to about 10 μm, having a uniform release profile after a storage for three months at 40° C. and 75% relative humidity.

The present invention provides immediate release pharmaceutical compositions for oral administration comprising micronized atovaquone particles and at least one drug, which are in solid dosage forms.

The present invention provides the solid dosage forms of the pharmaceutical composition comprising micronized atovaquone particles and at least one drug as granules, pellets, minitablets, tablets, capsules. The preferred solid dosage forms are tablets.

The present invention provides a method for the preparation of the solid dosage forms of the pharmaceutical compositions comprising micronized atovaquone particles and at least one drug.

The present invention provides a method for micronization of atovaquone, comprising dry milling or wet milling or the combination thereof, with the proviso that a microfluidizer is not employed as a milling process.

### DETAILED DESCRIPTION OF INVENTION

The present invention provides immediate release pharmaceutical compositions for oral administration comprising micronized atovaquone particles.

The present invention provides immediate release pharmaceutical compositions for oral administration comprising micronized atovaquone particles, wherein about 90% of the micronized atovaquone particles have a volume diameter between about 3 μm to about 10 μm.

The present invention provides pharmaceutical compositions comprising micronised particles of atovaquone with at least one drug, and one or more pharmaceutically acceptable carriers, wherein about 90% of the particles have a volume diameter between about 3 μm to about 10 μm, preferably between about 4 μm to about 8 μm.

The present invention provides a method for the preparation of micronized atovaquone particles comprising wet milling or dry milling or a combination thereof.

The methods of micronization are not limited to milling but may include other conventional size reduction techniques known in the art, with the proviso, however, that the micronization techniques of the processes described herein do not include a microfluidizer or variations thereof.

The particle size of micronised atovaquone particles can be measured by conventional methods known in the art.

Conventional pharmaceutical formulations include formulations suitable for oral administration. Preferable solid dosage forms may include granules, pellets, minitablets, tablets, capsules; more preferably tablet forms.

The present invention provides a pharmaceutical composition comprising micronised atovaquone particles with at least one drug and one or more pharmaceutically acceptable carriers; and further comprise other pharmaceutical excipients, solubilizers, buffering agents, stabilizing agents, binders, lubricants, disintegrants, complexing agents, etc. In the present invention, carriers or diluents are selected from starch, saccharides, microcrystalline cellulose, other cellulose derivatives, calcium phosphate, sodium calcium phosphate (NaCaPO₄), sugar alcohol, lactose and mixtures thereof.

The solubilizer/wetting agent may include ionic and nonionic surfactants, and/or complexing agents. The wetting/solubilizing agents are selected from sodium lauryl sulfate and Tween®, polyethylene glycol, poloxamer, preferably poloxamer.

The stabilizers for the purpose of this invention include the excipients used to retard or check crystal growth, which may represented by a non-limiting example, such as polyvinylpyrrolidone.

The pharmaceutical compositions, herein described can be provided as solid dispersions/solid solutions.

Binders, which are agents used to impart cohesive properties to the pellets or granules may be water soluble and/or insoluble binders, preferably water soluble binders. The water soluble binders are selected from cellulose derivatives, sugars, gums, gelatin, povidone, pregelatinized starch, sugar solution, and polyvinyl alcohol. The most preferred water soluble binder is hydroxypropyl methylcellulose (HPMC) of different viscosity grades. The preferred viscosity grades of HPMC are about 3 cps to about 10 cps.

Disintegrating agents, which are the substances or mixtures of substances added to a pellet to facilitate its breakup or disintegration after administration, are typically modified or unmodified starches, clays, cross-linked PVP, low-substituted hydroxypropyl cellulose, and other modified or unmodified celluloses.

Further, it is also beneficial, but not necessary, that the dosage form can further comprise a small amount of a lubricant such as, magnesium stearate.

The choice of excipients is not limited to the example disclosed herewith; it may include any suitable excipients mentioned in the Handbook of Pharmaceutical Excipients (edited by Raymond Rowe, Paul Sheskey and Sian Owen, 5^{th} ed.; publishes by the Pharmaceutical Press and the American Pharmacists Association 2006).

The dosage form of the pharmaceutical compositions, herein described, can be prepared by tablet press, roller compactor, extruder or any other machine or machine aid used to prepare solid dosage forms, known in the art.

The present invention provides immediate release pharmaceutical compositions for oral administration comprising micronized atovaquone particles and at least one drug, wherein about 90% of the atovaquone particles have a volume diameter between about 3 μm to about 10 μm, having a uniform release profile after a storage for three months at 40° C. and 75% relative humidity.

The following examples are provided to enable one skilled in the art to practice the invention and are merely illustrative of the invention. The examples should not be read as limiting the scope of the invention as defined in the claims. The percent w/w formula compositions of the examples of the present invention are set forth as a table, in Example 1.

It will be understood that various modifications may be made to the embodiments disclosed herein. Therefore the above description should not be construed as limiting, but merely as exemplifications of preferred embodiments. For example, the functions described above and implemented as the best mode for operating the present invention are for illustration purposes only. Other arrangements and methods may be implemented by those skilled in the art without departing from the scope and spirit of this invention. Moreover, those skilled in the art will envision other modifications within the scope and spirit of the claims appended hereto.

### EXAMPLES

### Example: I

| | | |
|---|---|---|
| | | Qty/Tab. |
| S. No. | Ingredient | (mg) |

Granulation stage

| | | |
|---|---|---|
| 1. | Atovaquone | 250.00 |
| 2. | Proguanil hydrochloride | 100.00 |
| 3. | Microcrystalline cellulose | 35.50 |
| 4. | Low Substituted Hydroxypropyl | 20.00 |
| | Cellulose (L-HPC LH-21) | |
| 5. | Sodium starch glycolate | 50.00 |
| 6. | Poloxamer ®188 | 3.00 |
| 7. | Povidone | 25.00 |
| 8. | Purified water | qs |
| 9. | Microcrystalline cellulose (MCC) | 30.00 |
| 10. | Colloidal silicon dioxide | 2.50 |
| 11. | Magnesium Stearate | 4.00 |

Coating: Using Opadry ® Brown 03B86943, consisting of the following

| | | |
|---|---|---|
| 12. | HPMC 2910/Hypromellose 6 cps | 7.93 |
| 13. | Titanium dioxide | 3.28 |
| 14. | Macrogol ®/PEG 400 | 0.79 |
| 15. | Iron oxide Red | 0.74 |
| 16. | Macrogol ®/PEG 8000 | 0.25 |

Process:

Granulate the uniform mixture of atovaquone, proguanil hydrochloride, microcrystalline cellulose, low substituted hydroxypropyl cellulose, sodium starch glycolate with aqueous binder solution of poloxamer and povidone. Dry and shift the granules, blend the shifted granules with MCC, sodium starch glycolate and colloidal silicon dioxide. Lubricate the blend with magnesium stearate and compress into tablet of suitable weight and size. The compressed tablets were coated with Opadry® coating material.

### Dissolution Studies

The dissolution studies were carried out by using USP dissolution apparatus II. The dissolution media was 900 ml, 40% isopropyl alcohol buffered to pH 8 with potassium dihydrogen phosphate. Dissolution was performed at 37.0±0.5° C. with stirring at 50 rpm.

The results of the dissolution studies, tabulated below, indicate that the composition, prepared according to the process herein described, has a similar dissolution profile as that of a marketed composition. The composition herein described was tested after storage for three months at 40° C. and 75% relative humidity.

Dissolution Results:

| | | % release | | % release | |
|---|---|---|---|---|---|
| | Time in | of marketed composition | | of Example 1 composition | |
| S. No. | Minutes | Atovaquone | Proguanil | Atovaquone | Proguanil |
| 1. | 0 | 0 | 0 | 0 | 0 |
| 2. | 5 | 3 | 5 | 3 | 4 |
| 3. | 10 | 14 | 16 | 15 | 14 |
| 4. | 15 | 30 | 34 | 31 | 31 |
| 5. | 30 | 72 | 80 | 79 | 85 |
| 6. | 45 | 94 | 98 | 95 | 101 |

We claim

1. An immediate release pharmaceutical composition comprising, micronized atovaquone and at least one other active ingredient, wherein about 90% of the atovaquone particles have a volume diameter between at least about 4 μm to about 8 μm.

2. The composition of claim 1, where proguanil hydrochloride is the other ingredient.

3. (canceled)

4. A pharmaceutical composition comprising (a) atovaquone, in an amount of about 50 mg to about 300 mg, wherein about 90% of the atovaquone particles have a volume diameter between about 4 μm to about 8 μm, (b) proguanil hydrochloride, in an amount of about 25 mg to about 100 mg, (c) microcrystalline cellulose (d) low substituted hydroxypropyl cellulose, (e) poloxamer and (f) one or more pharmaceutically acceptable excipients.

5. The composition of claim 4, with a characteristic of releasing not less than about 60% of atovaquone and proguanil within about 30 minutes in 900 ml of 40% isopropyl alcohol buffered to pH 8 with potassium dihydrogen phosphate at 50 rpm.

6. The composition of claim 4, which is an immediate release unit dosage form that further comprises polymeric film coating.

7. The composition of claim 4, wherein the excipients include binder, stabilizer, disintegrant, and lubricant.

8. A process of preparing an immediate release pharmaceutical composition comprising atovaquone and proguanil hydrochloride, comprising: a) granulating a mixture comprising micronised atovaquone, wherein about 90% of the atovaquone particles have a volume diameter between about at least 4 μm to about 8 μm, proguanil hydrochloride, microcrystalline cellulose, low substituted hydroxypropyl cellulose and optionally other pharmaceutical excipients, with a granulation liquid; b) drying the wet granules and lubricating the dried granules; c) compressing the lubricated granules into tablets.

9. The process of claim 8, wherein the granulation liquid comprises an aqueous solution of binder, optionally a solubilizer.

10. The process of claim 9, wherein the binder is povidone.

11. The process of claim 9, wherein the solubilizer is poloxamer.

12. The composition as in claim 1, in the form of a pharmaceutical tablet.
